(19) United States
(12) Patent Application Publication
(10) Pub. No.: US 2017/0022536 A1
(43) Pub. Date: Jan. 26, 2017

(54) URINE METABOLITE MONITORING DEVICE AND MANAGEMENT SYSTEM

(71) Applicant: EIDO INNOVA, INC., Brownsville, TX (US)

(72) Inventors: Carlos Francisco Bernal Velazquez, Brownsville, TX (US); Nancy Guerra, Brownsville, TX (US)

(21) Appl. No.: 14/994,649

(22) Filed: Jan. 13, 2016

Related U.S. Application Data
(60) Provisional application No. 62/194,666, filed on Jul. 20, 2015.

(51) Int. Cl. C12Q 1/54; A61B 10/00

(52) U.S. Cl. CPC C12Q 1/54; A61B 10/007

(57) ABSTRACT

A simple and painless device and management system for measuring and analyzing levels of metabolites in urine. The portable, electronic device is easily placed inside a toilet and can wirelessly transmit the collected and synthesized data to smart mobile devices and alert other individuals of dangerous levels detected by the device. The device is self-cleaning and requires no manipulation by the user.

### CROSS-REFERENCES TO RELATED APPLICATIONS

This application claims the benefit of U.S. Provisional Application No. 62/194,666, filed Jul. 20, 2015, which is incorporated herein by reference in its entirety.

### FIELD OF THE INVENTION

The present invention is generally directed toward an improved device and management system for measuring metabolites in urine.

### BACKGROUND OF THE INVENTION

There are different ways of measuring metabolite levels in blood, urine, saliva, and tears with subcutaneous implanted needle-type sensors, etc. Some of them are portable, and others are not, and some do not even have circuit boards. In the case of glucose determination, the most common glucose meters are those that use blood to test glucose levels, as they are the most popular and affordable in the market. However, their readings require a complex procedure, as well as a painful prick to take a blood sample, which usually leaves the injection site very sensitive for hours and even days.

To operate the complex blood glucose meters known in the art many steps must be followed: 1. Wash or sanitize your hands; 2. Place a new needle in the needle-holder; 3. Place the test strip in the glucose meter, 4. Perform a fingerstick to get the sample; and 5. Collect the drop of blood on the test strip. Without mentioning that it is a painful procedure, several surveys indicate that up to 67% of diabetic patients do not measure their glucose levels as frequently as recommended by the World Health Organization, arguing mainly that the measurement procedure is painful and complicated. (Study by the Diabetes Care Journal).

In the case of urine test strips, the procedure is as follows: 1. Get a clean container to collect the sample; 2. Urinate into the container; 3. Dip the test strip into the urine; 4. Read the results according to the color pattern; and 5. Wash the container. This procedure may be disgusting for patients as they have to manipulate the sample and because of the odor of urine. In addition, the reading may not be objective, as it depends on different colors, which may be easily misinterpreted.

Another option for urine glucose measurement is Tanita UG-201, publication number US2006/0094124 Narushi, et al. With this device, the procedure is: 1. Get a clean container to collect the sample; 2. Urinate into the container; 3. Dip the device into the sample; 4. Wash the container; 5. Wash the device; and 6. Fill the device with a cleaning solution. In addition to performing this long procedure, this device is not able to show graphic data or make emergency calls.

Finally, there is a system developed by TOTO Ltd, U.S. Pat. No. 5,730,149A, launched in Japan in December 1993, by which the patient just goes to the toilet as usual and the measurement is taken. The main reasons this device is not accessible to everyone are the following: it is very expensive ($2,000 to $5,000 US dollars) and requires the installation of the toilet, which can only be performed by a TOTO Ltd. certified technician.

Therefore, a great need exists for a simple and painless procedure that makes the urine measurement and analysis process easier and more efficient for patients. Moreover, a need exists for a system that allows health care providers and caretakers to utilize the urine analysis data to monitor and improve the overall health and lifestyle of patients.

### SUMMARY OF THE INVENTION

The presently disclosed invention is an improved device and management system that painlessly and simply measures metabolites in urine. The portable electronic device can be placed inside the toilet and records, synthesizes and transmits data to an end user application. The device also has a self-cleaning mechanism and an alarm feature, capable of calling the hospital or alerting other individuals if life threatening metabolite levels are detected by the measurement.

### BRIEF DESCRIPTION OF THE DRAWINGS

Further advantages of the invention will become apparent by reference to the detailed description of preferred embodiments when considered in conjunction with the drawings:

FIG. 1 depicts a top plain view of the device mounted on the toilet.

FIG. 2 depicts a side plain view of the interior part of the device.

FIG. 3 depicts a side plain view of the exterior base of the device.

FIG. 4 depicts a side plain view of the circuit board and light sensor located in the interior part of the device.

FIG. 5 depicts a flow chart of the system operated by the device.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

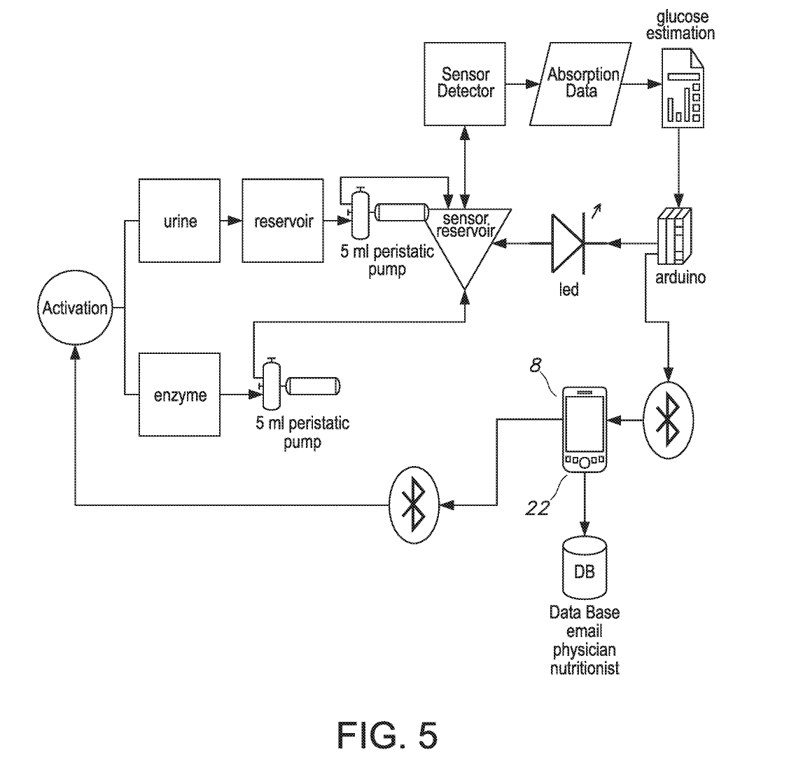

The improved electronic device 1 and management system claimed herein allows a user to painlessly and simply measure metabolite levels in their urine after simple installation in a toilet bowl. The user must only ensure that the urine stream is directed into the device so that a small amount of urine is collected by the device. As shown in FIG. 5, the user activates the device 1, and once the measurement has been taken, the results are synthesized and sent to the user. The device also has an alarm feature capable of alerting the hospital or user's caretakers if life threatening metabolite levels are detected by the measurement.

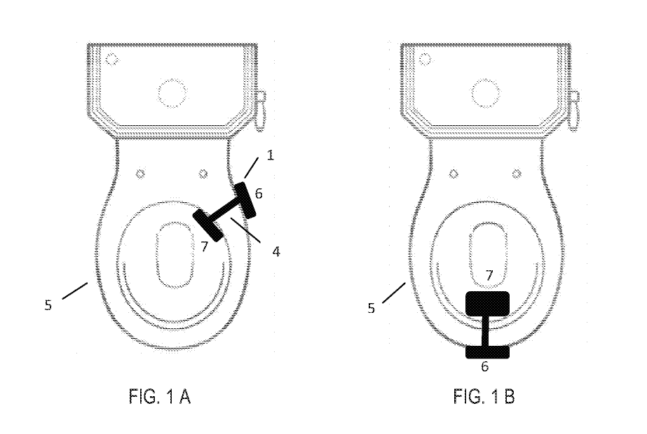

As shown in FIG. 1, the device, comprised of an interior part 2 placed inside the toilet and an exterior base 3 placed outside of the toilet connected by a bridge 4, is placed in two different positions inside a toilet bowl 5 depending on the sex of the user. For a male user, the device is placed on one of the sides of the toilet, and for a female user, the device is placed at the front of the toilet in order to collect the urine sample. The exterior base 3 of the device sits on the exterior ring of the toilet bowl 6, while the interior part 2 of the device connects to the inside of the toilet bowl 7.

As shown in FIG. 5, before using the device 1, the user must activate the device, including but not limited to, a phone or pad. Once enabled, a timer is activated and the user is given a limited amount of time to urinate into the interior part of the device 2.

In the preferred embodiment of the system, the user activates the device through a smart mobile device 8.

Other potential embodiments of activating the device include, but are not limited to, wireless means (Bluetooth, RFID, Ultrasound, Laser, Infrared, NFC, Xbee, Cellphone network, SMS), Voice, Internet, Button, Finger scan, etc.

The preferred embodiment of the invention allows the user 59 seconds to urinate.

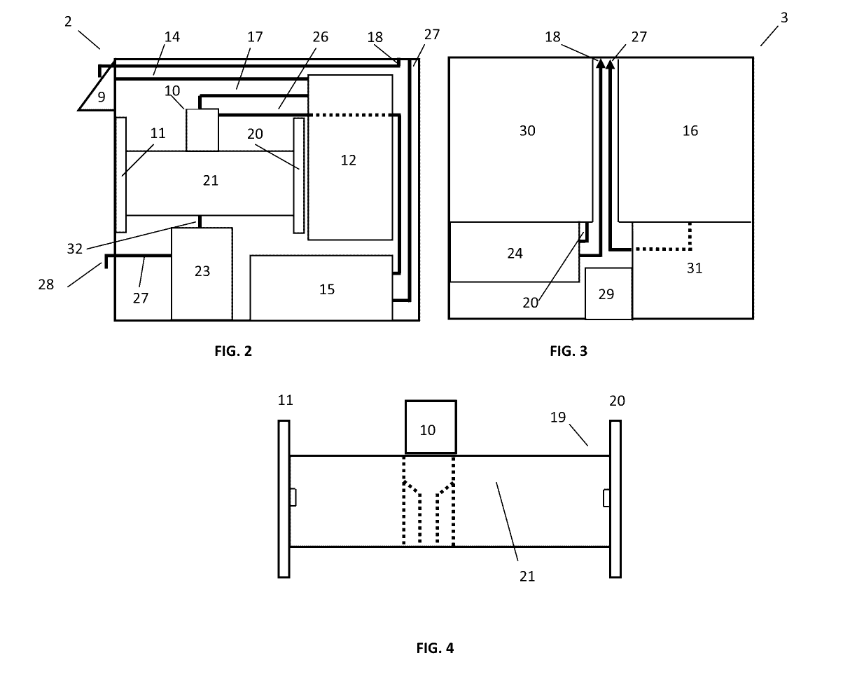

As shown in FIG. 2, the interior part of the device 2 further comprises a sample entry 9, container 10, circuit board 11, pumps, hoses and a drain valve 13. Also, as shown in FIG. 3, the exterior base of the device 3 further comprises a water container 30, reagent container 16, pump, electronic board with microcontroller 31, and battery 29.

After the allowed time frame has passed, the first pump 12 activates and collects a sample of urine from the sample collector 10 which is transferred via a first pump 12 through a first 14 and second hose 17 to the container 10 where the chemical reaction takes place and the measurement is performed. Once the urine has been supplied, a second pump 15 activates to introduce a reagent from the reagent container 16 via a third hose 26 and fourth hose 18 into the sample container 10.

The preferred embodiment of the sample containing approximately five microliters of urine.

The preferred embodiment of the device 1 measures glucose levels in urine using enzymatic colorimetry.

The preferred embodiment of the reagent comprising 500 microliters of Glucose Oxidase-Peroxidase. Also, the preferred embodiment of the reaction takes place in ten seconds and results in a reddish liquid.

Other potential embodiments of the device measure other metabolites in urine, including but not limited to, Urobilinogen, Bilirubin, Ketones, Blood, Albumin, Proteins, Nitrite, Leukocytes, Ascorbic Acid, Calcium, Creatinine, Cholesterol and Triglycerides, by reacting with the corresponding enzyme for each metabolite necessary to complete the reaction and result in an accurate measurement.

Another potential embodiment of the device includes measuring metabolite levels in urine by spectroscopy. In this embodiment, the device also includes a diffraction grating slide and a CCD camera.

The reaction takes place resulting in saturation proportional to the metabolite concentration in the urine. The preferred embodiment of the ratio of metabolite to enzyme ranging from 1:1 to 1:1000.

After the reaction takes place, the electronic circuit board with the microcontroller 31 and light sensor 20 starts the sample measurement by analyzing light absorption and transmission. The circuit board 11, mainly comprised of an LED emitting light, passes the light through the sample container 10 into the light sensor 19 on the circuit board 20. The light sensor 19 measures the amount of light reaching the end of the channel 21 and the microcontroller 31 calculates the amount of metabolite in the sample according to this amount. The higher the metabolite level, the lower the light passing through the sample. Different frequencies of light are used to differentiate the metabolite from other compounds that may be present in the urine.

When colorimetry is used to measure the metabolite levels, the LED specifically emits green, white, red and blue light. First, the red light is activated, then the green one, then the blue one and finally the three colors are combined to make a white light, which is directed through the channel 21. This operation is performed for each light color while the light sensor 20 measures the amount of light passing through the sample. The microcontroller 31 calculates average values for each light color analyzed.

The averages are compared against the calibration values and the microcontroller calculates transmittance and absorbency average for each color analyzed. These results are compared to a calibration curve to calculate the milligrams of metabolite per deciliter in the sample.

As shown in FIG. 5, the microcontroller 31 sends the results, which can be used to constantly monitor metabolite levels in the urine and detect various patterns and trends, including but not limited to, dietary patterns, that trigger such levels, to the application on the mobile device 8, which shows the results and stores them in the database 22.

Potential embodiments of the device including numerous methods of activating the device and transferring the results, including but not limited to, wireless means (Bluetooth, Infrared, NFC, Xbee, Cellphone network, SMS), Voice, Led, Internet, Display, etc.

Other potential embodiments of storing the information, including but not limited to, storing the information in a separate database, the cloud, and physical media.

The management system also includes an alarm function wherein synthesized data can be used to alert the hospital or the user's caretakers if dangerous metabolite levels are reported. Further, the data can assist the caretakers and health care providers with the necessary follow-up care and overall monitoring of the user's health.

Other potential embodiments of methods of using the information include pairing with Electronic Health Records (EHR) and data mining.

Once the measurement is completed, the cleaning process takes place. First, the drain valve 23 is activated to release the rest of the sample, then the pump 24 is activated and it supplies water into the sample collector for its cleaning; this water goes through the first hose 14 driven by the first 12 and second pump 15, through a third hose 26, then through the sample container 10 and through the fifth 32 and sixth hoses 27, and is finally disposed of into the toilet through the outlet hose 28.

The preferred embodiment of the cleaning process uses 500 microliters of water.

The preferred embodiment of the cleaning process receives water refills from a water tank. A potential embodiment of the cleaning process receives water from the toilet.

Other potential embodiments of the cleaning process use varying quantities of water, other cleaning liquids, or non-liquids, such as pressurized air.

After the cleaning process, the system returns to standby mode, with a battery 29 supplying the whole system.

The preferred embodiment of the device is powered by a 9V battery.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We hereby claim:

1. A portable device for analyzing the presence of metabolites in urine comprising:
a. a sample collector;
b. a reservoir for containing a reagent;
c. means for calculating quantities of the metabolites; and
d. an electronic communication module.

2. The device of claim 1 wherein said means for calculating quantities of said metabolites includes reagents mixing with the urine in the sample container, emitting light and detecting the transmittance and absorbency of each light color passing through the mixture to determine the amount of metabolites in the sample.

3. The device of claim 1 wherein said metabolite is glucose.

4. The device of claim 1 wherein said metabolite is selected from the group consisting of: urobilinogen, bilirubin, ketones, albumin, blood, proteins, nitrite, leukocytes, ascorbic acid, calcium, creatinine, cholesterol, and triglycerides.

5. The device of claim 1 wherein said device is capable of being activated by software on a handheld device.

6. The device of claim 5 wherein said handheld device is a smartphone.

7. The device of claim 1 wherein said electronic communication module sends an alert. In the event of an abnormal metabolite level.

8. The device of claim 1 wherein said device is Internet enabled.

9. The device of claim 1 wherein said device is placed inside a toilet bowl.

10. The device of claim 1 wherein said device is place outside a toilet bowl and the sample collector is placed inside the toilet bowl.
